(19) United States
(12) Patent Application Publication
(10) Pub. No.: US 2020/0221818 A1
(43) Pub. Date: Jul. 16, 2020

(54) APPARATUS AND METHOD FOR PLANTAR FASCIITIS

(71) Applicant: Paul David Edwards, Fullerton, CA (US)

(72) Inventor: Paul David Edwards, Fullerton, CA (US)

(21) Appl. No.: 16/360,460

(22) Filed: Mar. 21, 2019

Related U.S. Application Data
(60) Provisional application No. 62/792,079, filed on Jan. 14, 2019.

(51) Int. Cl. A43B 7/14

(52) U.S. Cl. CPC A43B 7/141; A43B 7/144; A61F 5/14; A43B 7/1445; A43B 7/143

(57) ABSTRACT

An apparatus and method for treatment of plantar fasciitis. The apparatus includes an insole for wear within a shoe, or footwear, of a patient afflicted with plantar fasciitis. The insole has an opening, or void, defined in a top surface of the insole such that the opening is positioned below a locus of the plantar fasciitis injury. The opening is positioned so that the insole does not impart a contact pressure about the point of injury. The method includes opening a space in the top surface area of an insole under the Plantar Fascia ligament tear to immediately relieve the pressure from the insole that causes Plantar Fascistic pain.

### CROSS-REFERENCE TO RELATED APPLICATION

This application claims the benefit of priority of U.S. provisional application No. 62/792,079, filed Jan. 14, 2019, the contents of which are herein incorporated by reference.

### BACKGROUND OF THE INVENTION

The present invention relates to plantar fasciitis, and more particularly to apparatus and methods of alleviating pain associated with the condition.

Plantar fasciitis is one of the most common causes of heel pain and has been estimated to affect about two million people in the US, resulting in more than one million visits to both primary care physicians and foot specialists. One challenge in the treatment of plantar fasciitis is that very few high-quality studies exist comparing different treatment modalities to guide evidence-based management. They found that most of the treatments were unpredictable or minimally effective. There are no medications other than pain relievers for the injury. Because there is such limited success of the treatment options available, physicians are unclear as to which option to pursue.

The plantar fascia is a ligament that is attached to the bottom (plantar) aspect of the heel bone (calcaneus). It begins as a narrow band in the center of the heel and then widens as it extends on into the long bones and toes. It is a thick, “bowstring-like” ligament that acts as a shock absorber for the arch of the foot. The ligament is both flexible enough to move the foot up and down. It is strong enough to cover and protect nerves, blood vessels and the muscles of the foot.

When the plantar fascia is overstretched it can lose its resiliency and result in painful micro-tears to the ligament that are called plantar fasciitis. Plantar fasciitis foot pain is caused by pressure around the tear in the plantar fascia ligament. The wound itself does not cause the pain, rather the pressure when standing/walking on the wound causes the pain.

Half of all heel pain is caused by plantar fasciitis. Half of all plantar fasciitis tears are close to where the ligament attaches to the front half of the heel bone. Diagnosis is important. Heel pain can be caused by plantar fasciitis, a bone spur, or an Achilles tendon among other things. They are treated in different ways. An x-ray can diagnose plantar fasciitis but not identify where the tear is located. An Ultrasound, CT or an MRI scan can pin point the location of the tear. While there are no definitive treatments for this condition, planta fascia under good conditions heals itself, sometimes in six months to a year.

Current solutions use padding and gels on top and bottom of the insole to reduce the pressure and relieve some of the pain.

As can be seen, there is a need for an apparatus and method for that can efficiently and effectively treat the inflammatory pain symptoms related to plantar fasciitis.

### SUMMARY OF THE INVENTION

In one aspect of the present invention an insole for the treatment of a plantar fasciitis wound is disclosed. The insole is dimensioned to be received within a shoe of a patient afflicted with plantar fasciitis. A void is defined in a top surface of the insole, wherein the void is positioned beneath a locus of the plantar fasciitis wound. In some embodiments, a heel end of the insole is dimensioned to support a majority of the afflicted patient's heel.

In some embodiments, the void further includes an opening extending between a top surface and a bottom surface of the insole. The void may be laterally disposed about a longitudinal centerline of the insole for alignment with the injury. The void may also be radially disposed about the longitudinal centerline of the insole from a point proximal to a heel end of the insole.

In other aspects of the invention, a method of treating a plantar fasciitis injury is disclosed. The method includes providing an insole for wear within a shoe of a patient afflicted with plantar fasciitis. The insole has an opening defined in a top surface of the insole that is positioned below a locus of the plantar fasciitis injury.

The method also includes inserting the insole into the patient's shoe. Other steps of the method include imaging the patient's foot to diagnose the plantar fasciitis injury and determining a locus of the plantar fasciitis injury. The method may also include positioning the opening about a lateral centerline of the insole to position the opening for an injury locus oriented towards one of an instep and an outstep of the patient's foot. The method may also include aligning the opening so that it is radially offset from the longitudinal centerline of the insole.

These and other features, aspects and advantages of the present invention will become better understood with reference to the following drawings, description and claims.

### BRIEF DESCRIPTION OF THE DRAWINGS

FIG. 1 is a perspective view of the invention with shoe removed for clarity;

FIG. 2 is an exploded view of the invention;

FIG. 3 is a section view of the invention, taken along line 3-3 in FIG. 1;

FIG. 4 is a top view of the insole;

FIG. 5 is a perspective view of the alternate embodiment with shoe removed for clarity;

FIG. 6 is an exploded view of the alternate embodiment;

FIG. 7 is a section view of the invention, taken along line 7-7 in FIG. 5; and

FIG. 8 is a top view of the insole.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

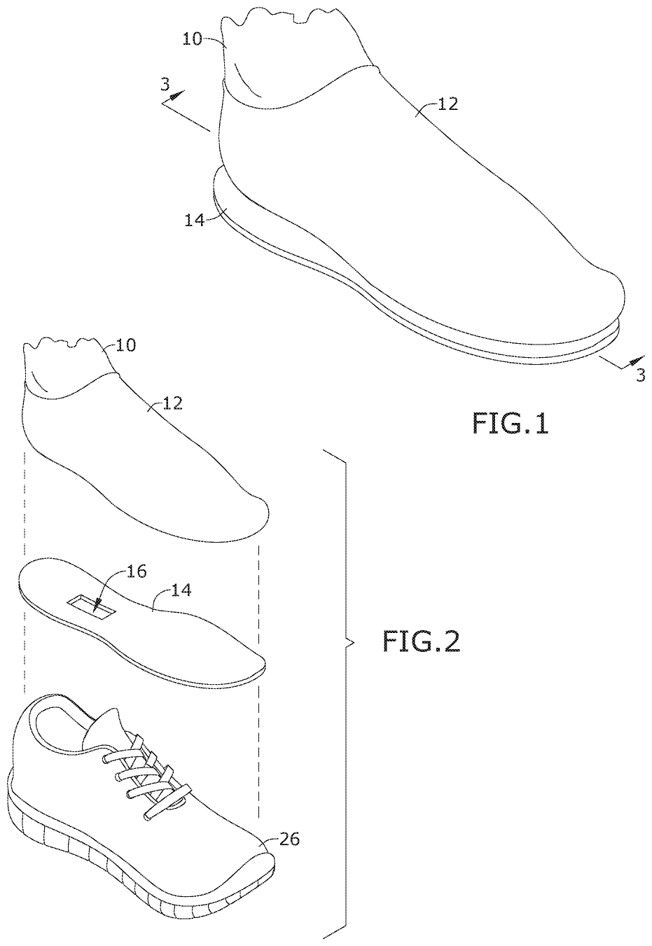

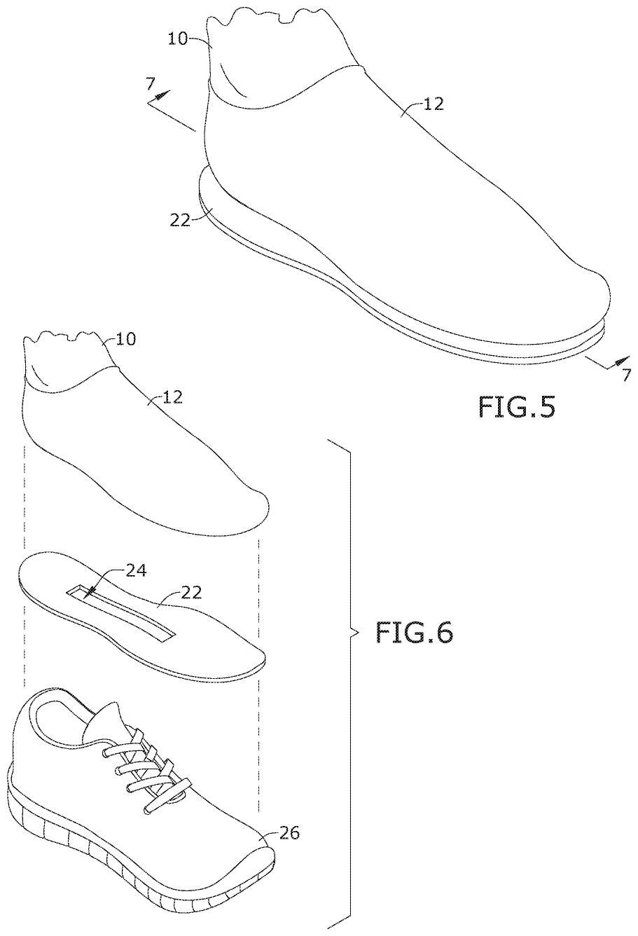

Broadly, embodiments of the present invention provides an apparatus and method for the treatment of plantar fasciitis. An insole according to aspects of the invention is shown in reference to the drawings of FIGS. 1-8. In FIGS. 1 and 2, a foot 10 of a patient afflicted with plantar fasciitis is shown with the patient wearing a sock 12. The insole 14 of the present invention is worn within a shoe 26.

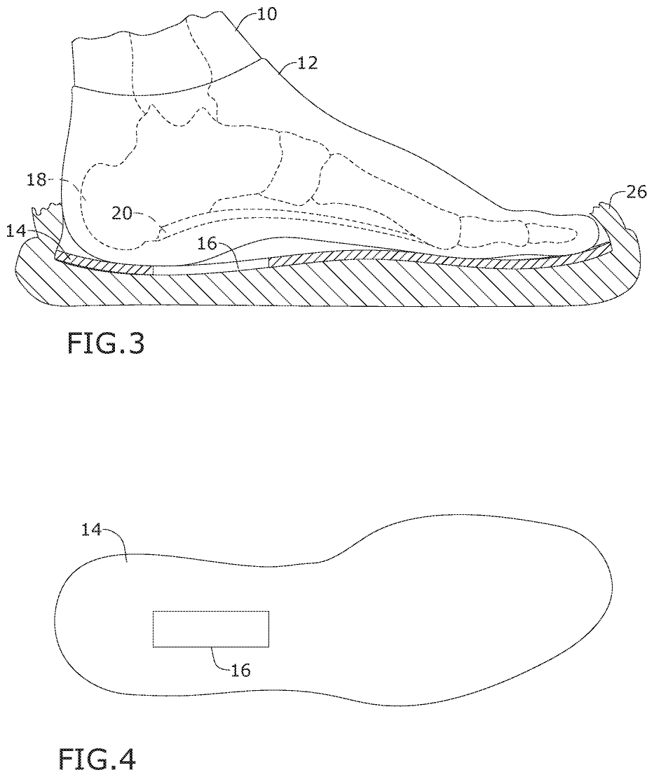

The insole 14 has an opening 16, or a void defined in a top surface of the insole 14 so that the opening 16 is positioned subjacent to the wound site 20. In the embodiment shown in reference to FIG. 3, the wound 20 is located proximal to the attachment point of the plantar fascia with the patient's heel 18. The opening 16 is then defined so that it is positioned subjacent to the wound site 20. The aft, or heel end of the insole 14 is positioned in a supporting relation with a majority of the heel bone 18 along a longitudinal axis of the insole 14. With a majority of the heel bone 18 thus supported, the heel bone 18 remains elevated so that the wound site 20 is not contacted by the insole 14. The opening 16 may also be defined in a lateral dimension of the insole 14 so that it is positioned subjacent to the locus of the injury 20, such as towards the instep, outstep, or medial portion of the heel.

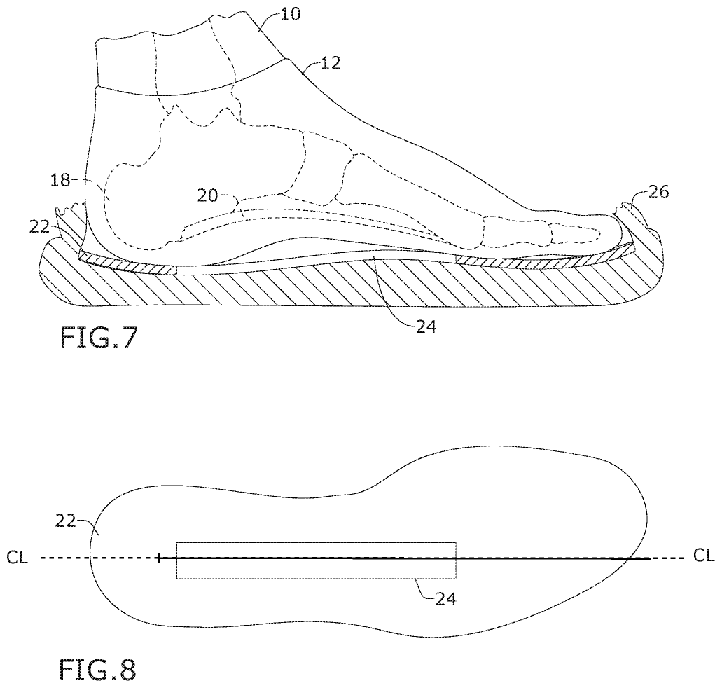

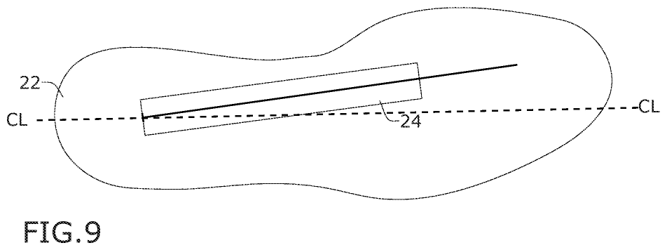

As seen in reference to the embodiment illustrated in FIGS. 7 and 8, with the wound 20 located towards an anterior portion of the arch, the opening 24 in the insole 22 may be elongated such that the void 24 is subjacent to the injury site 20. While the opening 24 is illustrated along a centerline of the insole 22, the opening 24 may also be displaced laterally in the insole 22 to accommodate for a locus of the injury 20 that is oriented along an outstep or an instep of the patient's foot 10. With an anterior injury, the locus of the wound 20 may also vary based on the particular metatarsal bone the plantar fascia connects. Accordingly, a longitudinal orientation of the opening 24 may be radially displaced from the longitudinal centerline of the insole 22, between the heel 18 and the wound 20.

A method of treating plantar fasciitis may thus include, providing an insole 14 for wear by an afflicted patient, wherein the insole 14 has an opening 16 defined in the insole 14 subjacent to the locus of the plantar fasciitis injury 20. The opening 16 of the insole 14 thus eliminates the insole pressure on the wound 20. Without the insole pressure on the wound 20, pain is thereby relieved. The open space can be used for any plantar fasciitis ligament tear.

The method may further include, inserting the insole 14 into the patient's shoe. Earlier steps in the method may include imaging the patient's foot to diagnose a plantar fasciitis condition, and the locus of the plantar fasciitis injury 20. The method may further include positioning the opening 16 about a lateral centerline of the insole 14 to position the opening 16 for an injury locus oriented towards one or more of an instep and an outstep of the patient's foot. Yet other method steps may include disposing the opening 16 so that it is radially offset from the longitudinal centerline of the insole 14.

It should be understood, of course, that the foregoing relates to exemplary embodiments of the invention and that modifications may be made without departing from the spirit and scope of the invention as set forth in the following claims.

1. An insole for the treatment of a plantar fasciitis wound, comprising:
an insole dimensioned to be received within a shoe of a patient afflicted with plantar fasciitis, the insole configured to extend between a heel and a toe end of the shoe, a heel end of the insole is configured to support a majority of a heel of the afflicted patient; and
a void defined in a top surface of the insole, wherein the void is configured to be positioned beneath a minority of the heel of the afflicted patient and a locus of the plantar fasciitis wound such that the locus of the plantar fasciitis wound is not contacted by the insole.

2. (canceled)

3. The insole of claim 1, wherein the void further comprises an opening extending between a top surface to a bottom surface of the insole.

4. The insole of claim 1, wherein the void is laterally disposed about a longitudinal centerline of the insole.

5. The insole of claim 4, wherein the void is radially disposed about the longitudinal centerline of the insole from a point proximal to a heel end of the insole.

6. A method of treating a plantar fasciitis injury, comprising:
providing an insole adapted for wear within a shoe of a patient afflicted with plantar fasciitis, a heel end of the insole is configured to support a majority of a heel of the afflicted patient, the insole having a void defined in a top surface of the insole the void configured to be positioned beneath a minority of the heel of the afflicted patient and below a locus of the plantar fasciitis injury.

7. The method of claim 6, further comprising:
inserting the insole into the patient's shoe.

8. The method of claim 6, further comprising:
imaging the patient's foot to diagnose the plantar fasciitis injury; and
determining the locus of the plantar fasciitis injury from the image of the patient's foot.

9. The method of claim 6, further comprising:
positioning the opening about a lateral centerline of the insole to position the opening for an injury locus oriented towards one or more of an instep and an outstep of the patient's foot.

10. The method of claim 6, further comprising:
aligning the opening so that it is radially offset from the longitudinal centerline of the insole.

11. An insole for the treatment of a plantar fasciitis wound, consisting of:
an insole adapted to be received within a shoe of a patient afflicted with plantar fasciitis, the insole configured to extend between a heel and a toe end of the shoe, a heel end of the insole is configured to support a majority of a heel of the afflicted patient; and
a void defined in a top surface of the insole, wherein the void is configured to be positioned subjacent to a minority of the heel of the afflicted patient and a locus of the plantar fasciitis wound such that the locus of the plantar fasciitis wound is not contacted by the top surface of the insole.
